(19) United States
(12) Patent Application Publication
(10) Pub. No.: US 2021/0338457 A1
(43) Pub. Date: Nov. 4, 2021

(54) ENVELOPING BODY AND METHOD FOR DETECTING A CONTOUR OF AN AMPUTATION STUMP

(71) Applicant: OTTOBOCK SE & CO. KGAA, Duderstadt (DE)

(72) Inventors: Erik ALBRECHT-LAATSCH, Rosdorf (DE); Michael NOLTE, Seeburg (DE); Jens DRIESEBERG, Clausthal-Zellerfeld (DE); Christian WILL, Göttingen (DE); Sebastian BETZ, Göttingen (DE); Hendryk ENGELBART, Göttingen (DE); Lars Benjamin FINKE, Landolfshausen (DE); Thilo-Mathias BIELEFELD, Siedenlangenbeck (DE)

(73) Assignee: OTTOBOCK SE & CO. KGAA, Duderstadt (DE)

(21) Appl. No.: 17/284,431

(22) PCT Filed: Oct. 4, 2019
(86) PCT No.: PCT/EP2019/076862

§ 371 Date: Apr. 9, 2021

(30) Foreign Application Priority Data
Oct. 11, 2018 (DE) 10 2018 125 144.0

(51) Int. Cl. A61F 2/50; A61F 2/76; A61F 2/78; G01B 15/04

(52) U.S. Cl. CPC A61F 2/5046; A61F 2/76; A61F 2002/7818; G01B 15/04; A61F 2002/762; A61F 2/7812

(57) ABSTRACT

The invention relates to an enveloping body for at least partially recording a contour of a limb, wherein the enveloping body has a base body, and at least one sensor that is configured to record measurement data which can be used to determine a distance and/or relative position between two points in or on the base body.

The invention relates to an enveloping body for at least partially detecting a contour of a limb, the enveloping body comprising a base body. The invention also relates to a method for at least partially detecting a contour of a limb with which such an enveloping body is used.

An enveloping body is understood in particular to mean prosthesis liners and bandages which at least partially, but preferably completely, enclose a limb or an amputation stump when mounted as intended. The limbs can be an upper limb. i.e. an arm, or a lower limb, i.e. a leg, but also a part of a torso of the wearer.

A prosthesis usually features a prosthesis socket into which the amputation stump is inserted. The prosthesis socket is usually made of a rigid material, such as a carbon fiber composite material. Especially in the case of leg prostheses with which the patient walks or runs, large forces act on both the prosthesis socket and the amputation stump. In order to avoid painful pressure points, it is very important that the prosthesis socket, which is usually custom-made for the wearer of the prosthesis, is adapted as effectively as possible to the shape of the amputation stump. However, there is the difficulty that the amputation stump has both bony parts that are particularly sensitive to pressure and therefore need to be protected and soft parts that deform under the strain of walking or standing. For this reason, it makes sense to compress the amputation stump, i.e. in particular to apply pressure to it, and to modify it with a special-purpose mould in order to at least partially detect the shape and contour of the prosthetic socket to be produced. This is called pre-compression.

The prior art comprises various methods for producing a prosthesis socket. For a long time, the standard method was to mould the amputation stump with plaster, for example. This has the advantage that, for example, the orthopaedic technician making the impression can apply pressure to the amputation stump with his hands and thus manipulate the shape of the residual limb in the sense of a special-purpose mould. A positive mould of the amputation stump is created from this negative impression, which is then used to manufacture the prosthesis socket. The disadvantage of this is that the procedure is laborious and uncomfortable for the patient, as the patient has to wait until the plaster has hardened and the plaster mould can be removed from the amputation stump.

In order to achieve a uniform pre-compression of the amputation stump during the moulding process, various methods are known from the prior art. For example, it is proposed to insert the amputation stump into a container that is filled with sand. In this way, a pressure can be applied by the patient themselves that corresponds to the pressure that will later occur when standing. A negative imprint of the amputation stump is thus created in the sand, which can be poured out, for example. Alternatively, it is proposed to insert the amputation stump in a flexible membrane into a water tank and use the pressure of the water for pre-compression. Inside the water tank, the amputation stump prepared in this way can, for example, be scanned without contact, using optical scanning, and in this way the contour can be recorded.

DE 20 2016 001 130 U1, on the other hand, discloses a device that has a fastening ring with a liner arranged on it into which the amputation stump is inserted. The advantage of this device is that a pressure distribution is exerted on the amputation stump by the liner into which the amputation stump is inserted, which largely corresponds to the pressure distribution exerted by the actual liner worn inside the prosthetic socket. In this liner too, which is held over the retaining ring or fastening ring, the amputation stump can then be scanned using non-contact scanning techniques.

From a press release of the Fraunhofer Institute for Silicate Research ISC from 2015, it is known in principle to integrate strain or pressure sensors made of silicone elastomers into textiles. The explanation is available at https://www.isc.fraunhofer.de/de/presse-und-medien/pressearchiv/pressearchiv2015/textilintegrierte-drucksensoren.html.

However, the disadvantage is that the pressure distribution cannot be adjusted manually, as the orthopaedic technician does, for example, with the plaster cast method. In addition, scanning the amputation stump in a water tank, for example, requires a lot of equipment. The invention thus aims to further develop a liner in such a way that the disadvantages of the prior art are eliminated or at least mitigated.

The invention solves the problem with an enveloping body according to the preamble in claim 1, characterized in that the enveloping body has at least one sensor that is configured to record measurement data which can be used to determine a distance and/or relative position between two points in or on the base body. Consequently, the idea according to the invention consists in already equipping the sensor in such a way that at least a part of the contour of the enveloping body can be determined by elements arranged in the enveloping body, in this case the at least one sensor, or at least corresponding measurement data are recorded with which this is possible. This significantly reduces the amount of equipment required to record the contour and also speeds up the procedure, which is particularly advantageous for the patient.

Preferably, the sensor has at least one transmitter for a measuring radiation and a receiver for the measuring radiation, which are arranged in such a way that measuring radiation emitted by the transmitter is at least partially received by the receiver. In this way, for example, the distance between the transmitter and the receiver can be determined via a time-of-flight measurement.

Preferably, a transmitter and a receiver are located at the first point, wherein a reflector for the measuring radiation is preferably located at the second point. The reflector does not have to be a separate component, for example in the form of a mirror, but can also be in the form of an interface between two different materials. This is advantageous, for example, when using ultrasound as measuring radiation that is reflected at interfaces. Such an interface is present, for example, where the material of the base body meets the limb of the wearer or patient. Depending on the measuring radiation used, an interface is characterised by a sufficiently large density jump of the two adjacent materials, a sufficiently large jump of the optical refractive indices of the adjacent materials or a jump of another physical or chemical quantity at the interface.

Alternatively, the transmitter of the sensor is preferably located at the first point and the receiver at the second point. Measuring radiation emitted by the transmitter at least partially strikes the respective receiver. If, for example, the intensity of the received measuring radiation is known at the receiver when the enveloping body is in the relaxed state without having been pulled over a limb, for example an amputation stump, it is possible to determine from the intensity received in the mounted state to what extent the material of the base body in which the measuring radiation is moving has been stretched. The longer the path between the transmitter of the measuring radiation and the receiver, the lower the received intensity of the measuring radiation at the receiver.

Alternatively or additionally, the time-of-flight of the measured radiation from the transmitter to the receiver can be determined. This can be used to determine the distance between the transmitter and receiver, provided that the speed at which the measuring radiation expands is known.

Alternatively or additionally, transmitter and receiver are located at the first point and a reflector for the respective measuring radiation is situated at the second point. The functional principles of the measurement are identical, but twice the distance is measured, which improves the signal-to-noise ratio.

Preferably, the measuring radiation is electromagnetic radiation, in particular visible light, radar radiation and/or X-rays. Particularly in the case of X-rays, it is possible to send the measurement radiation through the limb located in the enveloping body, even if this may have disadvantages for the patient's health. Alternatively or additionally, the measuring radiation can also be magnetic radiation, in particular in the form of an alternating magnetic field. In this case, the transmitter is an electromagnetic coil, i.e. an electrical conductor wound into a coil to which an alternating electrical current is applied. As a result, the coil acting as the transmitter, also known as the transmitting coil, generates an alternating magnetic field. The receiver preferably contains at least one receiver coil which is located in this alternating magnetic field. This results in the induction of an electric current and/or voltage in the receiver coil, which can be measured and depends on the distance between the transmitter coil and the receiver coil. In addition, the magnitude and phase of the induced electrical quantity in the receiver coil depend on the orientation of the transmitter coil to the receiver coil.

Therefore, it is advantageous if several coils, preferably perpendicular to each other, are used in particular as transmitters. These can be operated simultaneously with an electric alternating current. Depending on the phase relationship between the alternating current of the individual coils, a radiation direction in which the alternating magnetic field is primarily emitted can be influenced. It has been found to be advantageous if an alternating magnetic field is generated whose primary direction varies, preferably circulates cyclically. As a result, the magnetic field induced in the receiver coil is always minimal when the preferred direction or main transmitter direction in which the magnetic field with the maximum intensity is transmitted does not point to the respective receiver coil of the receiver.

In this configuration, it is therefore possible to detect the orientation of the receiver coil relative to the transmitter. To this end, it is advantageous if the initial orientation, i.e. the orientation of the transmitter relative to the receiver in the unloaded state of the enveloping body, i.e. without limb, is known.

Alternatively or additionally, the measuring radiation may also contain sonic waves. Ultrasonic waves are particularly suitable for this. In this case, too, the transmitter and receiver can be located at two different places, wherein, for example, the single distance, i.e. the distance between the two points, is measured via time-of-flight measurements. If the transmitter and receiver of the measuring radiation are in the same location, a reflector element is preferably arranged at the second point which reflects the measuring radiation. As previously explained, the reflector element does not have to be a separate component. Even when using sonic waves as measuring radiation, double the distance can be measured in this way, thus improving the signal-to-noise ratio.

The enveloping body preferably comprises several receivers and/or several reflectors. In a preferred configuration, the enveloping body has, for example, only one transmitter for the respective measuring radiation. In this case, however, the enveloping body has a plurality, preferably at least 10, preferably at least 20, especially preferably at least 50 or even at least 100, receivers and/or reflectors for the measuring radiation. In this way, the distances of many points, namely the points at which the receivers or reflectors are located, to the transmitter can be determined and ascertained. Since the arrangement of the individual receivers or reflectors relative to each other, i.e. in particular the neighbourhood relationships between the components, cannot change without destroying the enveloping body, the contour of the enveloping body can be determined from the distances to the different points. Alternatively or additionally, the sensor is configured to also determine a direction in which the respective receiver and/or the respective reflector are located. In this way, it is even easier to determine the contour of the prosthesis liner and thus the contour of the limb under the pressure of the enveloping body from the measurement data.

The transmitter is preferably located in the distal area of the enveloping body. i.e. the closed lower end of a prosthesis liner.

Preferably, the at least one sensor features a strain sensor, with which a distance between two points between which the sensor is arranged can be determined. The strain sensor is preferably at least one strain gauge, an electroactive polymer and/or a fiber Bragg element.

A strain gauge is understood particularly to mean a sensor in which the electrical resistance changes, for example within a metallic conductor track, as soon as the strain gauge is subjected to a strain. From the change in electrical resistance, conclusions can be drawn about the change in strain and thus about the distance between the two points at the ends of the strain gauge. An electroactive polymer, called an artificial muscle, changes its shape and especially its length when an electrical voltage is applied. However, such an electroactive polymer can conversely also be used as a sensor for a change in length, since the change in length of the electroactive polymer causes an electrical voltage. Since the relationship is also known here, the detectable electrical voltage can be used to infer the change in the length of the polymer and thus the change in the distance between its two ends.

A fiber Bragg element, on the other hand, is an optical component that uses a light guide with a material in its core that has varying refractive indices. These are so-called optical inscribed interference filters. These elements are also strain-sensitive, as the so-called Bragg wavelength, which corresponds to the center wavelength of the filter bandwidth, depends on a mechanical strain or mechanical stress. Here, too, a change in strain and thus a change in the distance can be inferred via the known relationship.

In a preferred configuration, the enveloping body features a plurality, preferably at least 10, preferably at least 20, especially preferably at least 50 or 100 strain sensors which are arranged in the material of the base body of the enveloping body in such a way that they are preferably equidistantly distributed across the limb once the enveloping body has been pulled over the limb. Due to the known neighbourhood relationships between different end points of the respective strain sensors, conclusions can also be drawn in this way about the contour of the enveloping body from the changed lengths of the strain sensors once the enveloping body has been pulled over the limb.

It is advantageous if at least one of the sensors is a shape sensor. Within the scope of the present invention, a shape sensor is understood to mean a sensor which has a length extension and is able to make statements not only about its length but also about its geometric shape via corresponding measurement data. Particularly preferred examples are sensors that have, for example, several layers of plastics arranged in a sandwich structure, if there is a change in the contour of such a contour sensor, for example in the form of bending, the individual layers are stretched or compressed to different degrees, which leads, for example, to a change in the thickness of the layer pack of the sensor. This is measured at different points so that different bends can be determined. Alternatively or additionally, so-called cable-like shape sensors can be used, such as those offered by the company TST-inno. Alternatively or additionally, light guides that feature a sufficient number of support points according to the fiber Bragg principle can also be used as shape sensors. At least three support points are sufficient, but more would be advantageous. The more support points there are and the more densely they are arranged, the more accurate the shape detection of the shape sensor designed in this way.

Preferably, the enveloping body features a communication interface by means of which the measurement data detected by the at least one sensor can be transmitted to an electronic data processing device, in particular a micro-processor. The desired information about the contour of the enveloping body can then be recorded from the measurement data.

In a preferred configuration, a corresponding electronic data processing device is already provided in or on the base body of the enveloping body, particularly at the distal end. In this way, only the enveloping body is required to detect the contour of the enveloping body and thus the contour of the limbs to the enveloping body, without having to use additional components and/or other devices. This further reduces the amount of equipment required to determine the contour.

The base body is preferably made from an elastic material. The enveloping body is preferably a prosthesis liner, preferably for a leg prosthesis, lower leg prosthesis or an upper leg prosthesis, wherein the base body is preferably made of a liner material. Liner materials are in particular silicone, polyurethane or TPE, wherein these materials can form the liner material individually or in combination with each other and/or in combination with a textile and/or a surface coating.

As an alternative to the prosthesis liner, the enveloping body can also be a bandage. With such a bandage, 3D models and 3D patterns for later manufacturing processes, for example CAM (Computer Aided Manufacturing), can be created by all processes in which optical scanners are already used today. This includes milling processes as well as additive processes, for example 3D printing processes. Contours and shapes recorded using a bandage can also be used for diagnostic purposes, for example comparing two different conditions.

The invention also solves the problem by means of a method for at least partially detecting a contour of a limb, wherein the method comprises the following steps:
mounting an enveloping body of the kind described here on the limb.
recording measurement data by means of the at least one sensor.
detecting distances and/or relative positions between two points in or on the base body in an electronic data processing device.

The shape of the limb is preferably manipulated during at least part of the process of recording the measurement data. This can be done manually or by a device.

It is especially preferable if distances and/or relative positions between at least 10 points, preferably at least 20 points, preferably at least 50 points and especially preferably at least 100 points are detected. The more distances and/or relative positions that can be detected, the more accurate the resulting image of the contour of the limb. The individual sensors can be connected in the form of a bus system, in a series or row or in any other way. Depending on the sensors, the individual sensors can be controlled simultaneously, consecutively in a specific order, cyclically or in any other way.

Preferably, continuous measurement data is recorded. Alternatively, the recording of the measurement data can be triggered when the enveloping body is deformed from the outside, i.e. in particular when a pressure from the outside acts on at least a part of the enveloping body.

In the following, some examples of embodiments of the present invention will be explained in more detail by way of the attached figures: They show:

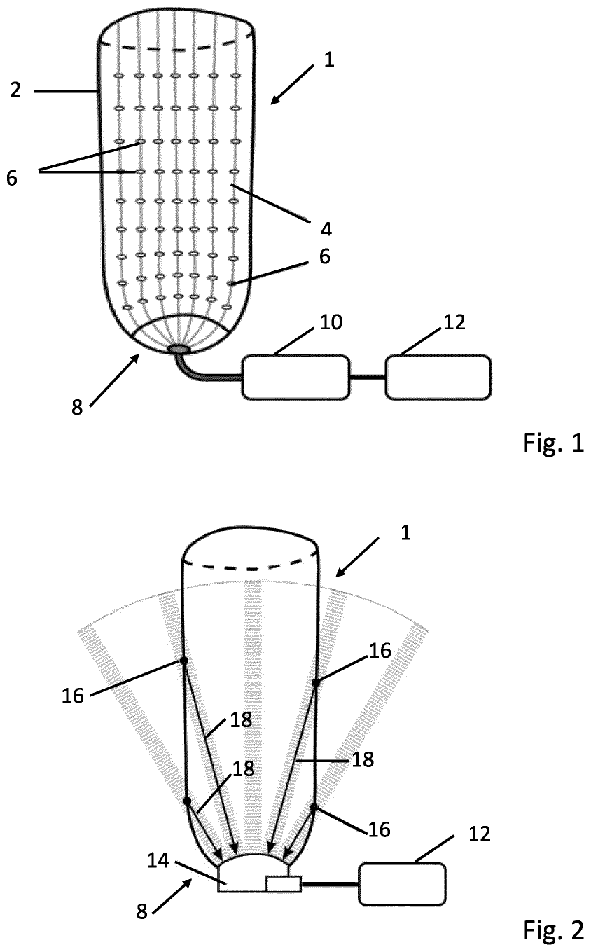

FIGS. 1 to 4—various embodiments of an enveloping body according to an example of an embodiment of the invention as a prosthesis liner, and

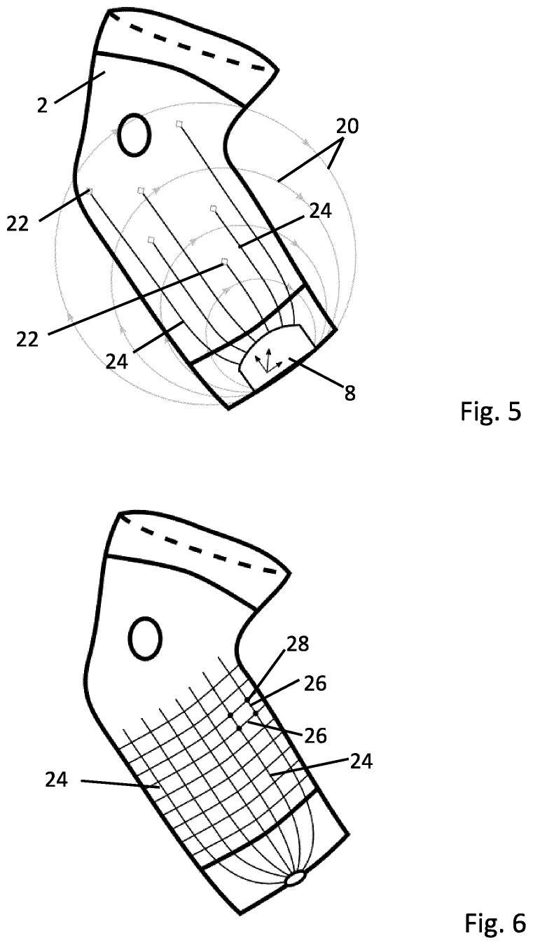

FIGS. 5 to 8—various embodiments of an enveloping body according to an example of an embodiment of the invention as a bandage.

FIG. 1 shows an enveloping body 1 according to a first example of an embodiment of the present invention in the form of a prosthesis liner. It has a main body 2 made of a liner material. The liner material contains several optical fibers 4 into which fiber Bragg sensors 6 are incorporated. The optical fibres 4 converge at a distal end 8 of the liner and can be exposed to light by an interrogator 10. This is preferably done one after the other. The individual fiber Bragg sensors 6 exhibit characteristic behaviour as optical interference filters. They absorb light of a certain wavelength, this wavelength depending on the strain or mechanical tension of the fiber Bragg sensor 6, as previously explained. In particular, in the event that the individual fiber Bragg sensors 6, which are interconnected by a single optical fibre 4, are designed differently and have different absorption spectra, they can be interrogated successively or even simultaneously. To this end, it is advantageous, for example, if the fiber Bragg sensors 6 that are closer to the interrogator 10 do not absorb electromagnetic radiation that is absorbed by fiber Bragg sensors 6 that are connected by the same optical fibre 4 but further away from the interrogator 10. If the fiber Bragg sensors are subjected to mechanical stress or strain, the center frequency in particular, which is also known as the Bragg frequency, is displaced in a known manner so that the strain can be inferred. Since the order of the individual fiber Bragg sensors 6 along the optical fibers 4 cannot change, the different strains can also be used to infer the contour of an amputation stump which is located within the prosthesis liner but is not shown in FIG. 1.

The interrogator 10 interrogates the individual fiber Bragg sensors 6 and transfers the measured data to an electronic data processing device 12 in which the actual evaluation takes place.

FIG. 2 shows a different configuration of the prosthesis liner 1. There is a transmitter 14 in the vicinity of the distal end 8 which emits a measuring radiation. In the example shown, these are ultrasonic waves. These are reflected by reflectors 16 and travel along arrows 18 back to the sensor, which also contains a receiver. The measurement data is then transferred to the electronic data processing device 12 and evaluated.

The advantage of ultrasonic waves in particular is that they are coupled by the transmitter 14 into the soft tissue of the amputation stump and expand in it. It is not necessary to ensure that the ultrasonic waves propagate through the liner material, which can be an elastic silicone material, for example. The transmitter 14 can, for example, transmit in continuous operation or emit its measuring radiation in the form of pulses. A rotating transmitter 14 can also be used, wherein the transmitter 14 itself does not necessarily have to rotate; rather, only the emitted measuring radiation is emitted in different angular ranges, which preferably rotate. The measuring radiation, i.e. the ultrasonic radiation in this example, strikes the reflectors 16 and is reflected back by them and reaches the receiver, which is not shown separately in FIG. 2. In this way, the distances of the individual reflectors 16 from the transmitter 14 can be determined via time-of-flight measurements. However, it is disadvantageous that ultrasonic signals in particular can be reflected at boundary surfaces and bony components of an amputation stump also provide a signal.

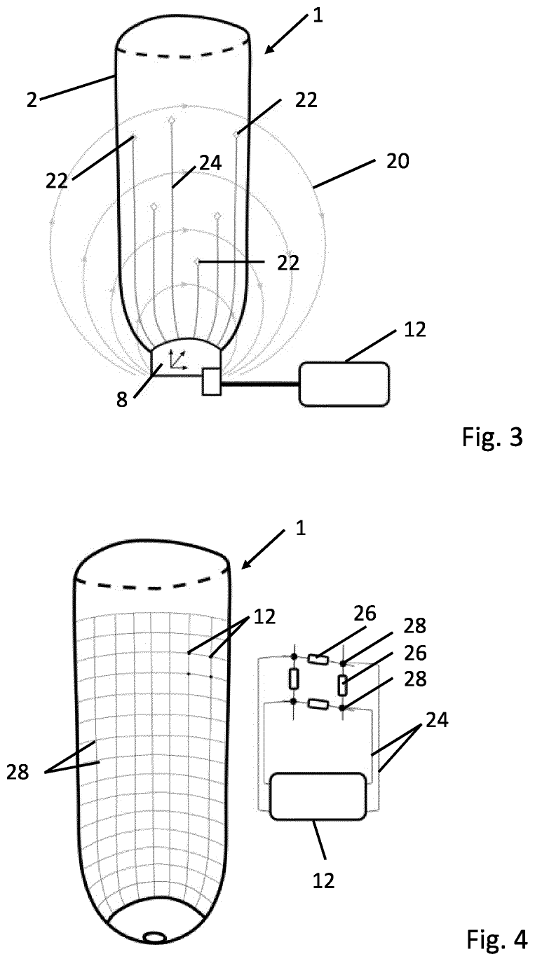

FIG. 3 shows another configuration of the prosthesis liner 1. There is a transmitting coil in the area of the distal end 8, said transmitting coil being supplied with alternating current and emitting an alternating magnetic field 20. This is also preferably designed to rotate, so that the main transmission direction of the transmitter 14 also changes. Inside the liner material of the base body 2 of the prosthesis liner are receivers 22 in the form of receiving coils, which are connected to the distal end 8 via electrical lines 24. The alternating magnetic field 20 induces an electric current and an electric voltage in the receivers 22, which can be tapped and measured via the electrical line 24. The magnitude and phase of the electric current and/or voltage induced in the receivers 22 depends on both the distance of the receivers 22 from the transmitter 14 and the orientation and direction of the receiver 22 relative to the transmitter 14. This measurement data is then transferred to the electronic data processing device 12 and evaluated.

FIG. 4 shows another embodiment of the prosthesis liner 1 in which strain sensors in the form of strain gauges 26 are arranged in the form of a mesh. In the example of an embodiment shown, they form a square grid with intersection points 28, wherein a strain gauge 26 is located between every two intersection points 28. An enlargement of this arrangement is shown in the right-hand area of FIG. 4. If the prosthesis liner is now pulled over an amputation stump, it stretches in some places more than others, depending on the shape of the amputation stump. Electrical lines 24, which are shown in the right-hand part of FIG. 4, can be used to apply electrical current and/or voltage to the individual intersection points 28. An electronic data processing device 12 controls this process. By applying an electrical voltage, the strain gauge 26 located between the two specified crossing points 28 is measured and the measurement signals thus obtained are evaluated by the electronic data processing device 12. Since the neighbourhood relations of the different strain gauges 26 and also the intersection point 28 are known and cannot change, a detailed picture of the contour of the prosthesis liner can be determined from the knowledge of the respective distances between two neighbouring intersection points 28. The denser and tighter the mesh, the more detailed and better the image of the contour.

FIGS. 5 to 8 depict various embodiments of the enveloping body 1 in the form of a bandage. The function of the bandage according to FIG. 5 corresponds to that of the prosthesis liner according to FIG. 3. The distal area again shows the transmitting coil that emits the alternating magnetic field 20. Again, there are receivers 22 inside the main body 2, which are connected to a distal end of the bandage via electrical lines 24. The alternating magnetic field 20 induces an electric current and an electric voltage in the receivers 22, which can be tapped and measured via the electrical line 24.

The function of the bandage according to FIG. 6 corresponds to that of the liner according to FIG. 4. The bandage shown in FIG. 6 also has a network of strain gauges 26 arranged between intersection points 28. Electrical wires 24 which cross at the intersection points can apply an electrical current or voltage to the individual strain gauges 26.

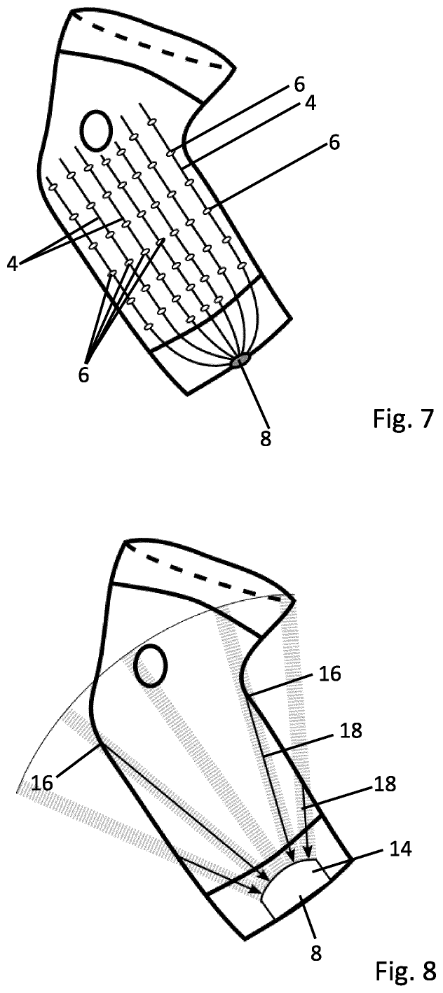

The function of the bandage according to FIG. 7 corresponds to the function of the prosthesis liner according to FIG. 1. Fiber Bragg sensors 6 are interconnected by optical fibres 4, which are brought together at a distal end 8.

The function of the bandage according to FIG. 8 corresponds to that of the liner in FIG. 2. The transmitter 14 is situated in the vicinity of the distal end 8, wherein said transmitter emits the measuring radiation, which in the example of an embodiment shown may be ultrasonic waves, for example. These are reflected along the arrows 18 by reflectors 16, which in the example of an embodiment shown represent interfaces between two different materials, and conveyed back to the transmitter. Since the transmitter also contains a receiver, measurement data can be evaluated and transferred.

### REFERENCE LIST

1 enveloping body
2 base body
4 optical fiber
6 fiber Bragg sensor
8 distal end
10 interrogator
12 electronic data processing device
14 transmitter
16 reflector
18 arrow
20 alternating magnetic field
22 receiver
24 electrical line
26 strain gauge
28 intersection point

1. An enveloping body for at least partially recording a contour of a limb, wherein the enveloping body has a base body, characterized in that the enveloping body has at least one sensor that is configured to record measurement data which can be used to determine a distance and/or relative position between two points in or on the base body.

2. The enveloping body according to claim 1, characterized in that the sensor has a transmitter for a measuring radiation and a receiver for the measuring radiation, which are arranged in such a way that the measuring radiation emitted by the transmitter is at least partially received by the receiver.

3. The enveloping body according to claim 2, characterized in that the transmitter and the receiver are located at a first point, the enveloping body further comprising a reflector for the measuring radiation which is located at a second point, or alternatively that the transmitter is located at the first point and the receiver at the second point.

4. The enveloping body according to claim 3, characterized in that the measuring radiation is electromagnetic radiation, in particular visible light, radar radiation and/or X-rays, magnetic radiation, for example in the form of an alternating magnetic field and/or sonic waves, such as ultrasonic waves.

5. The enveloping body according to claim 3, characterized in that the enveloping body comprises more than one receiver and/or more than one reflector.

6. The enveloping body according to claim 1, characterized in that the at least one sensor comprises a strain sensor, in particular a strain gauge, an electroactive polymer and/or a fiber Bragg element.

7. The enveloping body according to claim 1, characterized in that the at least one of the sensor is a shape sensor.

8. The enveloping body according to claim 1, characterized in that the enveloping body features a communication interface by means of which the measurement data recorded by the at least one sensor can be transmitted to an electronic data processing device, in particular a micro-processor.

9. The enveloping body according to claim 1, characterized in that the base body is made from an elastic material.

10. The enveloping body according to claim 1, characterized in that the enveloping body is a prosthesis liner and the base body is made of a liner material.

11. A method for at least partially recording a contour of a limb, wherein the method comprises the following steps:
mounting an enveloping body according to claim 1 on the limb,
recording measurement data by means of the at least one sensor,
determining distances and/or relative positions between two points in or on the base body in an electronic data processing device.

12. The method according to claim 11, characterized in that a manipulation of a shape of the limb occurs during at least a part of the process of recording the measurement data.

13. The method according to claim 11, characterized in that distances and/or relative positions between at least 10 points, at least 20 points, at least 50 points, or at least 100 points are detected.

14. The method according to claim 11, characterized in that the recording of the measurement data is continuous or triggered automatically when the enveloping body is deformed from the outside.

15. An enveloping body for at least partially recording a contour of a limb, wherein the enveloping body includes:
a base body;
at least one sensor; and
a communication interface;
wherein the sensor further comprises a transmitter and a receiver, the sensor being configured to record measurement data which can be used to determine a distance and/or relative position between first and second points in or on the base body of the enveloping body, and wherein the communication interface further comprises an electronic data processing device.

16. The enveloping body of claim 15, wherein the transmitter and the receiver are arranged on the enveloping body such that a measuring radiation emitted by the transmitter is at least partially received by the receiver.

17. The enveloping body of claim 16, wherein the transmitter and the receiver are located at a first point on the enveloping body, and wherein the enveloping body further includes a reflector located at a second point on the enveloping body.

18. The enveloping body of claim 16, wherein the measuring radiation emitted by the transmitter is electromagnetic radiation, visible light, radar radiation and/or X-rays, magnetic radiation, and/or sonic waves, such as ultrasonic waves.

19. The enveloping body of claim 15, wherein the enveloping body includes more than one receiver and/or more than one reflector.

20. The enveloping body of claim 15, wherein the at least one sensor is a strain sensor, such as a strain gauge, an electroactive polymer and/or a fiber Bragg element.
